(19) United States
(12) Patent Application Publication
(10) Pub. No.: US 2022/0411776 A1
(43) Pub. Date: Dec. 29, 2022

(54) PRO-THROMBIN PURIFICATION

(71) Applicants: Omrix Biopharmaceuticals Ltd., Rehovot (IL); Guangzhou Bioseal Biotech Co., Ltd., Guangzhou Science City (CN)

(72) Inventors: AIBIN YU, Guangzhou Science City (CN); JIANPING YU, Guangzhou Science City (CN); ISRAEL NUR, Nes-Ziona (IL); LIOR WEISSMAN, Nes-Ziona (IL); ITAI PODOLER, REHOVOT (IL)

(21) Appl. No.: 17/780,233

(22) PCT Filed: Dec. 3, 2019
(86) PCT No.: PCT/CN2019/122619

§ 371 Date: May 26, 2022

(51) Int. Cl. C12N 9/74

(52) U.S. Cl. CPC C12N 9/6429; C12Y 304/21005

(57) ABSTRACT

Provided is a method of purifying a protein of interest from a medium comprised of adsorbent and the protein, the method includes, inter alia, providing the medium of the protein, which is at least partially adsorbed into/onto the adsorbent, and performing pressure filtering to wash the adsorbent-adsorbed protein and/or to elute the protein from the adsorbent, thereby at least partially purifying the protein.

### FIELD OF THE INVENTION

The present invention relates, inter alia, to a method of protein purification e.g., purification of prothrombin from plasma using filter press.

### BACKGROUND OF THE INVENTION

Thrombin is a serine protease that facilitates blood clotting by catalyzing the conversion of fibrinogen to fibrin. Thrombin is also responsible for activating platelets and indirectly responsible for regulation of its own production and inhibition through multiple proteolytic feedback mechanisms. Thrombin is also involved in activation of factor VIII, factor V, factor XI, factor XIII and protein C. Thrombin is widely used in clinical applications as a coagulation factor to staunch bleeding of wounds by conversion of fibrinogen to fibrin, is a common component of surgical dressings, and has been used in combination with fibrinogen and other coagulation proteins in two-component hemostatic systems such as fibrin glues, adhesives, and sealants.

Thrombin is produced by proteolytic activation of the precursor (zymogen) prothrombin. For the production of thrombin, prothrombin must be cleaved at two sites generating intermediate products. The conversion of prothrombin to thrombin in the body is catalyzed by the prothrombinase complex which includes activated Factor X and Factor V, and assembles on negatively charged phospholipid membranes in the presence of calcium ions.

Thrombin may be manufactured from prothrombin by contacting a source of prothrombin (such as blood plasma or a blood fraction), with a solid adsorbent capable of adsorbing the prothrombin from the source of prothrombin, for example barium sulfate (BaSO₄). The solid adsorbent is typically washed using a washing solution to remove contaminants such as unbound proteins, and subsequently the prothrombin is eluted therefrom using an elution solution. Subsequent to additional optional purification and processing steps, the eluted prothrombin can be converted to thrombin by activation using an activator, e.g., calcium ions.

The current (referred to as “manual” or “non-filter press”) purification process of the prothrombin using barium sulfate binding includes manual washing to remove the impurities for three times, manual elution to elute the prothrombin from the barium sulfate for five times. Each step of washing and elution necessitates crushing barium sulfate by hand, centrifugation to separate the barium sulfate from buffer, and digging out the barium sulfate from the bottle of centrifugation.

### SUMMARY OF THE INVENTION

The invention relates, inter alia, to a method for purifying protein e.g., prothrombin from plasma using filter press.

The currently used process for binding proteins of interest from plasma, e.g., obtain the prothrombin from absorbed prothrombin on barium sulfate has some disadvantages such as manual operation, low production efficiency, carrying a high risk of contamination, repeated use of centrifuges which are time consuming. In addition, the current process is difficult to scale up. In embodiments of the method of the present invention a filter press is used, optionally as an automated process, an instead of manual washing, elution and/or centrifugation, does not require centrifugation, is easy to scale up, saves time, and reduces the risk of contamination, hence at least partially overcomes these disadvantages.

According to an aspect of the present invention, there is provided a method of purifying a protein of interest from a medium comprising insoluble adsorbent (e.g., barium sulfate (BaSO₄ or aluminium hydroxide)) reagent and the protein, the method comprising providing the medium comprising the protein being at least partially adsorbed into/onto the adsorbent (e.g., insoluble salt such as BaSO₄ or aluminium hydroxide), and performing pressure filtering to wash the adsorbent (such as e.g., BaSO₄ or aluminium hydroxide)-adsorbed protein (which is the retentate) and/or to elute the protein from the adsorbent, thereby at least partially purifying the protein.

Herein, protein being at least partially adsorbed into/onto the adsorbent is also denoted as: “adsorbent-adsorbed protein”.

In some embodiment, the step of performing pressure filtering is carried out by passing the medium in a pressure filter, e.g., a filter press.

In some embodiments, the adsorbent comprised insoluble salt. In some embodiments, the insoluble salt comprises aluminium hydroxide. In some embodiments, the insoluble salt comprises insoluble alkaline earth metal salt. In some embodiments, the insoluble alkaline earth metal salt is or comprises a BaSO₄ reagent.

In some embodiments, the medium comprises a source of the protein. In some embodiments, the medium is a liquid medium. In some embodiments, the protein comprises prothrombin. In some embodiments, the medium comprises a source of prothrombin. In some embodiments, the method comprises performing pressure filtering to wash the adsorbent (such as insoluble salt (e.g., BaSO₄ or aluminium hydroxide))-adsorbed protein and to elute the protein from the adsorbent (such as e.g., BaSO₄ or aluminium hydroxide). In some embodiments, the adsorbent (such as e.g., BaSO₄ or aluminium hydroxide)-adsorbed protein is washed using a washing buffer.

In some embodiments, the method comprises one or more steps selected from: (i) centrifuging the medium, thereby obtaining a sediment comprising the adsorbent (such as e.g., BaSO₄ or aluminium hydroxide) reagent and/or the protein; (ii) washing the protein being at least partially adsorbed into/onto the adsorbent reagent by a washing buffer, thereby removing therefrom impurities; and (iii) eluting a fraction comprising the protein from the adsorbent-adsorbed protein, using an elution buffer. In some embodiments, the method comprises the above-mentioned step (ii) and (iii), wherein at least one step of steps (ii) and (iii) is carried out by, or simultaneously to the step of passing the medium in a pressure filter.

In some embodiments, the adsorbent comprises an insoluble salt. In some embodiments, the adsorbent reagent is in the form of powder.

In some embodiments, the pressure filter is carried out by a filter press. In some embodiments, the protein is prothrombin and the source of prothrombin is selected from the group consisting of blood plasma or a plasma fraction. In some embodiments, the plasma comprises oxalated plasma. In some embodiments, the source of prothrombin comprises plasma harvested from a mammal. In some embodiments, the mammal is selected from the group consisting of a human, an equine, a bovine and a porcine. In some embodiments, the source of prothrombin comprises porcine plasma.

In some embodiments, the method comprises a step of contacting the adsorbent (such as insoluble salt (e.g., BaSO₄)) reagent and the source of prothrombin under conditions allowing adsorption of prothrombin from the source of prothrombin by the adsorbent (such as insoluble salt (e.g., BaSO₄)) reagent, thereby adsorbing prothrombin into/onto the adsorbent. In some embodiments, the conditions allowing adsorption of prothrombin from the source of prothrombin by the adsorbent (such as insoluble salt (e.g., BaSO₄)) reagent comprise a medium having pH ranging from 7.4 to 8.6.

In some embodiments, the step of performing pressure filtering comprises passing the medium through a filtration chamber under pressure, and the filtration chamber comprises filter membrane. In some embodiments, the pressure ranges from 1.5 to about 4 bar. In some embodiments, the step of performing pressure filtering comprises passing the medium in the pressure filter and exerting a back pressure onto the membrane, the back pressure ranging from 5 psi to 15 psi, thereby obtaining a uniform cake of the adsorbent (such as insoluble salt (e.g., BaSO₄))-adsorbed protein in/on the filter membrane. The term “back pressure” refers to a pressure in the direction opposite the flow direction of the medium.

The term “uniform” relates to essentially lacking (i.e. typically less than 10% or less than 5%) of variation, thickness or diversity.

In some embodiments, the filter membrane is characterized by a filtration capacity of at least 30 kg of the source of prothrombin per m².

In some embodiments, the medium comprises about 0.5 to 3% (w/w) adsorbent such as BaSO₄ reagent, optionally about 1%.

In some embodiments, the washing step is repeated 2 to 6 times.

In some embodiments, upon washing, the amount of proteins other than thrombin (e.g., fibrinogen) is reduced.

In some embodiments, upon washing, the medium comprises less than 0.5, less than 0.4, less than 0.3, less than 0.2, less than 0.1 mg/ml, or is even devoid of fibrinogen.

In some embodiments, proteins other than thrombin (e.g., fibrinogen) which has been washed away may be further purified.

In some embodiments, the washing buffer as added at weight ratio washing buffet-to-plasma ranging from 1:100 to 1:25. In some embodiments, the washing buffer is added at weight ratio washing buffet-to-plasma of 1:100, 1:75, 1:50, or 1:25, including any value and range therebetween. In exemplary embodiments, the washing buffer as added at weight ratio washing buffet-to-plasma of about 1:50.

In some embodiments, the washing buffer comprises sodium chloride and/or sodium citrate.

In some embodiments, the protein is eluted from the adsorbent (e.g., BaSO₄)-adsorbed protein using an elution buffer, thereby obtaining an eluted protein-containing fraction. The elution buffer (e.g., 200 ml) may be pumped into the filter press system and circulated e.g., for 5 to 30 min.

In some embodiments, the elution buffer as added at weight ratio washing buffet-to-plasma ranging from 1:100 to 1:25. In some embodiments, the elution buffer as added at weight ratio washing buffet-to-plasma of 1:100, 1:75, 1:50, or 1:25, including any value and range therebetween. In exemplary embodiments, the elution buffer as added at weight ratio elution buffet-to-plasma of about 1:50.

In some embodiments, the elution buffer comprises a calcium chelating salt, optionally is at pH of about 6.3 and 7.4. In some embodiments, the calcium chelating salt comprises sodium citrate. In some embodiments, the concentration of sodium citrate ranges from about 3% (w/v) to about 4.4% (w/v). In some embodiments, the method further comprises a step of concentrating the eluted prothrombin-containing fraction.

In some embodiments, the method further comprises a step of diafiltrating the eluted protein-containing fraction in a diafiltration buffer. In some embodiments, the diafiltration buffer comprises glycine. In some embodiments, the diafiltrating step is repeated 2 to 6 times.

In some embodiments, the protein is prothrombin, and the method further comprises a step of providing conditions which allow conversion of the prothrombin, into thrombin, thereby obtaining a thrombin. Additionally, or alternatively the eluted protein-containing fraction may be lyophilized.

In another aspect, there is provided a method of obtaining a thrombin from a source of prothrombin, the method comprising: (i) passing a liquid medium comprising adsorbent, optionally BaSO₄ reagent, and a source of the prothrombin in a pressure filter, thereby at least partially separating and/or purifying the prothrombin from the medium, and (ii) providing conditions which allow conversion of the prothrombin into thrombin, thereby obtaining a thrombin.

In some embodiments of any aspect, the adsorbent, optionally BaSO₄ reagent, at least partially adsorbs the prothrombin. In some embodiments of any aspect, the conditions which allow conversion of prothrombin into thrombin comprise subjecting the prothrombin to an activator such as calcium ions.

In some embodiments of any aspect, the thrombin is present in a fraction, and the method comprises a step of passing the thrombin containing fraction in a filter to remove therefrom micro floc.

In some embodiments of any aspect, the method is characterized by obtaining a thrombin yield of 70 to 130 IU per 1 ml of source of prothrombin, optionally plasma, within 4 hours.

As used herein, the term “IU” denotes “International Units” and may be determined by the clotting assay against an internal reference standard for potency concentration measurement that has been calibrated against, for example, the World Health Organization (WHO) Second International Standard for Thrombin, 01/580. A unit (U) is equivalent to an International Unit (IU).

In some embodiments, there is provided thrombin obtained by the method of any aspect provided herein. In some embodiments, the thrombin is characterized by activity of 4000 to 6000 IU/ml. In some embodiments, the thrombin is characterized by specific activity of 700 to 1200 IU per mg protein.

Further embodiments of the aspect method of obtaining a thrombin from a source of prothrombin, are provided hereinabove with regards to the aspect of the method of purifying a protein of interest, and form an integral part of embodiments relating to the aspect method of obtaining a thrombin.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-B present a flow chart outlining of a non-limiting example of the disclosed filter press process for purifying prothrombin (the washing and/or the elution may be carried out by filter press; FIG. 1A); and additional steps (starting from the eluate) made, according to a non-limiting example, to concentrate the eluate and to obtain thrombin from the purified prothrombin (FIG. 1B);

FIG. 2 presents a photographic image showing barium sulfate precipitates cake on the filter membrane after washing and eluting prothrombin;

FIGS. 3A-B present results of SDS PAGE coomassie staining (FIG. 3A) and Western blot (FIG. 3B) for prothrombin and thrombin in different fractions from the lab scale study compared with prothrombin and thrombin standards; the data series 1-9: 1—α,β,γ thrombin standards, 2—MW markers, 3—Prothrombin standard, 4—plasma, 5—unbound plasma, 6— wash fraction, 7—eluted fraction, 8—Activated for 24 h, and 9—Activated for 61 h; “*” denotes prothrombin, “**” denotes thrombin;

FIGS. 4A-D present bar graphs visualizing the characteristics of thrombin obtained using a filter press process of the invention followed by activation of prothrombin to thrombin (three trials) vis-à-vis a non-filter press process (“current process”), based on Table 7 below: protein content (mg/ml; FIG. 4A); thrombin activity (IU/ml; FIG. 4B); thrombin yield (IU/ml plasma; FIG. 4C), and specific enzyme activity IU/mg protein; FIG. 4D); and

FIG. 5 presents a scatter diagram of thrombin activity (received from using filter press process and activation of thrombin) frozen under −20° C. (Y-thrombin activity in IU/ml vs. X-days the thrombin kept frozen).

### DESCRIPTION OF EMBODIMENTS OF THE INVENTION

An object of the present invention is to provide an improved process of purification of proteins e.g., prothrombin.

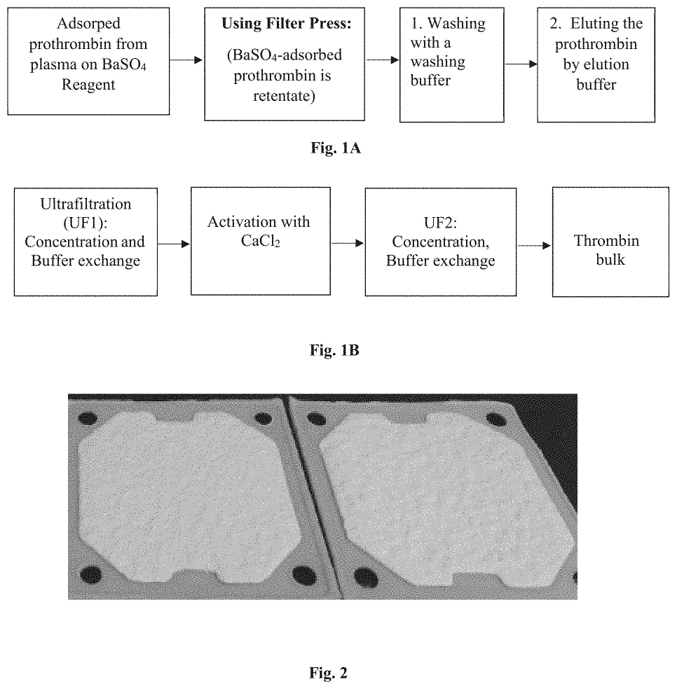

As explained below, the non-filter press process of purification comprises the following steps: Binding (mixing barium sulfate with plasma (1% mg/mg); Separation by centrifugation to separate barium sulfate from plasma and collect barium sulfate cake; Washing using washing buffer, crashing the sediment into small pieces by manual, followed by centrifugation to separate solid from liquid (this step is typically repeated 3 times to remove impurity); and Elution by adding elution buffer followed by crashing the sediment into small pieces by manual, further followed by centrifuging to separate solid from liquid. This elution step is typically repeated 5 times to collect prothrombin complex. Since centrifugation is used for the solid/liquid separation, precipitates collected from the centrifuge need to be crashed to small pieces by hand, in order to easily wash/elute. As washing step is typically repeated three times, elution step is typically repeated five times, in total, centrifugation is used 9 times including the plasma removal step. Thus, this process is very complex, the contamination risk is higher, and typically takes 16 hours to complete the all operation. Reference is made to FIG. 1A presenting a flow chart outlining of a non-limiting example of the disclosed method (also referred to as “the filter press process”).

The disclosed process, in some embodiments thereof, may be used to replace at least one, and optionally two, or even the three of the above-mentioned steps of: (i) centrifugation e.g., for plasma removal; (ii) the washing carried out manually and/or by centrifugation, and (iii) elution carried out manually and/or by centrifugation.

The suspension (e.g., BaSO₄+plasma) of the disclosed process, in some embodiments thereof, is separated by the depth filter on the filter press. The adsorbent (e.g., BaSO₄) may hold by the filter to form (e.g., as a retentate) a thin cake layer of e.g., prothrombin-BaSO₄, and, in some embodiments, in the next operation, washing buffer is used to flush the adsorbent (e.g., BaSO₄) cake by a pump. After this, the liquid may be replaced by elution buffer. The prothrombin may be thereafter eluted from adsorbent (e.g., BaSO₄) cake. These steps of washing/elution operation in the disclosed process are also referred to as “online” washing or elution, respectively, that is, being performed during the filter pressing without the need of manual operation as in the non-filter press process. Typically, the total process time may be reduced to e.g., 4 hours or less. Accordingly, the methods described herein in some embodiments thereof, are quick and simple to use, and potentially provide saving of time and/or production costs. As used herein, the term retentate refers to the solid fraction e.g., a slurry which remains on the filter

In some embodiments, the total time duration of the disclosed process (up to washing and/or elution steps) is within less than 16 hours, less than 15 hours, less than 14 hours, less than 13 hours, less than 12 hours, less than 11 hours, less than 10 hours, less than 9 hours, less than 9 hours, less than 8 hours, less than 7 hours, less than 6 hours, less than 5 hours, or less than 4 hours, or, in some embodiments, is within 1 to 4 hours, or within 2 to 4 hours from the onset of the process.

The term “cake” refers to composition, typically, but not exclusively, in the form of a porous or spongy structure like layer or film having some water content, typically the water content not being visible. By “porous”, it is meant that the material at and under the surface is permeated with interconnected interstitial pores or cavities that may communicate with the surface.

Accordingly, in one aspect of the present disclosure, there is provided a method for separating and/or purifying a protein of interest (e.g., prothrombin) from a medium comprising a filter aid material which may be an adsorbent such as, for example, insoluble salt, e.g., Al(OH)₃, and/or alkaline earth metal salt such as barium sulfate (BaSO₄) reagent and the protein, the method comprising providing the medium comprising the protein being at least partially adsorbed into/onto the an adsorbent (e.g., comprising BaSO₄ reagent), and performing pressure filtering, for example, by passing the liquid medium comprising the an adsorbent (e.g., comprising BaSO₄ reagent) and the source of the protein in a pressure filter, e.g., to wash the adsorbent-adsorbed protein and/or elute the protein from the an adsorbent, thereby at least partially purifying the protein.

In some embodiments, the term “filter aid” or “filter aid material,” refers to those materials which may conventionally be deposited on a filter screen or the like in order to aid in the filtration which is produced by the filter. In some embodiments, the filter aid comprises adsorbent, (also referred to herein as “sorbent”, “adsorbent material”, adsorbing reagent” or “adsorbing agent”).

The term “adsorbent” relates to one or more water insoluble solid particles, comprising insoluble materials which can adsorb to one or more proteins onto a surface of the particles. The term “adsorbent” is used herein for convenience of description and is used without intention to limit to any particular mechanism by which protein of interest may be taken into or onto a body of the water insoluble solid particles, and is not limiting as to different types of interaction that may occur with a adsorbent and protein being sorbed or adsorbed, which may include various chemical, molecular, atomic, or surface interactions as well as simple permeation, and optionally swelling of the adsorbent. In some embodiments, the adsorbing mechanism, generally refers, without limitation, to a surface phenomenon wherein an analyte becomes reversibly associated with a sorbent, typically the surface of the adsorbing agent, e.g., sorbent, by physically interacting with the surface molecules. The association may be, for example, via any non-covalent mechanism (e.g., van der Waal's forces, such as dipole-dipole interactions, dipole-induced dipole or dispersive forces, via hydrophobic interactions or hydrogen donor or acceptor interactions).

Non-limiting examples of adsorbent may comprise silicates (e.g., granite, basalt, and shale), carbonates (e.g., limestone and dolomite), and evaporites (e.g., halite).

Further adsorbent may be selected from diatomaceous earth, perlite, glass beads, magnesium silicate, calcium silicate, solid thermoplastic or thermoset polymer beads, and calcium silicate.

Typically, but not exclusively, the adsorbent comprises insoluble metal salt. The term “metal salt” refers to a compound comprised of at least one anion and at least one metallic (e.g., alkaline earth metallic) cation. The term “insoluble salt” means a metal salt that is completely or partially insoluble in a solution. In some embodiments, this term refers to water-insoluble salt, that is, a salt that is completely or partially insoluble in water at about room temperature.

In some embodiments, the insoluble salt comprises a sulfate salt, such as barium sulfate, calcium sulfate, and/or ammonium sulfate. In some embodiments, the alkaline-earth metal salt includes, but is not limited to, calcium carbonate, magnesium carbonate, calcium phosphate. In some embodiments, the insoluble salt comprises aluminium hydroxide, Al(OH)₃. In some embodiments, the alkaline-earth metal salt comprises BaSO₄.

In some embodiments, the pressure filtering is carried out to wash the adsorbent—e.g., BaSO₄— adsorbed protein and to elute the protein from the adsorbent, e.g., BaSO₄.

In some embodiments, the medium comprises a source of the protein.

As used herein, the term “analyte” means any molecule of interest, e.g., a protein such as prothrombin. An analyte may be disposed in a sample, such as a source of protein.

The term “purify” means increase concentration of a desired ingredient (up to 100 wt %), decrease concentration of one or more undesired ingredients (down to 0 wt %), or both.

The term “separating” means to increase the amount of one component in a sample (e.g., the protein of interest), relative to the amounts of other components in the sample.

As used herein, the term “protein” is used to refer to a polymer or an oligomer of amino acid residues. Herein, the term “protein(s)” also encompasses peptide(s).

In some embodiments, the protein is or comprises prothrombin. Prothrombin is a plasma protein involved in the final stages of blood coagulation, as well-known in the art. It has a molecular weight of about 72,000 and contains about 12% carbohydrate. Prothrombin is a calcium-binding protein that undergoes a conformational transition in the presence of calcium as is known. The proteolytic activation of prothrombin to thrombin is a critical step in normal hemostasis. Prothrombin is synthesized in the liver where a prothrombin precursor undergoes post-translational modification to yield the functional form of prothrombin which is known as “native prothrombin” and contains γ-carboxyglutamic acid.

Herein, the term “prothrombin” is further meant to encompass, in some embodiments, prothrombin complex. The term “prothrombin complex” is referred to as a mixture or solution of prothrombin with other one or more factors involved in blood coagulation, including e.g., blood coagulation Factor VII, Factor IX, Factor X, and the like.

In some embodiments of the methods disclosed herein, the source of prothrombin is selected from plasma (such as oxalated plasma) or a plasma fraction. In some such embodiments, the source of prothrombin comprises plasma harvested from a mammal, such as, without limitation, a human, an equine, a bovine and a porcine. In some embodiments, the source of prothrombin comprises porcine plasma. In some embodiments, the source of prothrombin is or comprises recombinant prothrombin. In some embodiments, the source of prothrombin is subjected to viral inactivation treatment. For example, the source is solvent/detergent (SD) treated plasma.

Normal mammalian plasma, such as human plasma, is a well-known pooled or single donor plasma preparation intended for use as a calibration plasma for various coagulation tests.

Normal human plasma may be sterile plasma obtained by pooling the liquid portion of whole blood to which has been added a solution of potassium or sodium citrate, or both, e.g., from eight or more healthy adult humans and by exposing it to ultraviolet light to destroy bacterial and viral contaminants Normal human plasma may be e.g., Unicalibrator calibration Plasma for Coagulation Tests 00625. By “sterile” it is meant essentially or even completely free from bacteria or other microorganisms such as viruses.

The term “filtration” includes all of those separation processes as well as any other processes utilizing a filter that separates one.

In some embodiments, prothrombin complex may be prepared by various procedures, including treatment of plasma with an anion exchanger to prepare prothrombin complex, production of prothrombin from cryoprecipitate-poor plasma which may be prepared by removing cryoprecipitate from plasma, and the like. Starting plasma may also be derived from sources of any animal species, including bovine or human, typically human.

The term “pressurized filter” or “pressure filter” refers to a filter being disposed such that there is a difference in pressure between two points or selected spaces in the filter; for example, between one side of a flow of the mixture and another side of the flow of the mixture passing mixture in the filter.

In some embodiments, the pressure filter is carried out using a filter press, for example, by passing a liquid medium comprising BaSO₄ reagent and a source of protein, e.g., prothrombin in the filter press.

The term “filter” refers to a device, typically having porous medium whose primary function is the separation and retention of particulate contaminants from a fluid.

The term “filter press” means a machine or device using filtering membrane or plates to separate solids and liquids by applying an external pressure typically through a permeable filter. The separation process may take place in chambers formed between every two filter plates. In this case, the solid phase is inside the chambers (forming a so-called “cake”), and the liquid phase (filtrate) penetrates through the filter media and flows out through the discharge ports.

In some embodiments, the protein is least partially adsorbed into/onto the adsorbing agent, e.g., BaSO₄.

In some embodiments, adsorbing the protein, e.g., the prothrombin, may be carried out by contacting an adsorbing agent, e.g., BaSO₄ reagent and the source of protein (e.g., prothrombin) under conditions allowing adsorption of protein (e.g., prothrombin) from the source of protein (e.g., prothrombin) by the adsorbing agent, e.g., BaSO₄ reagent, thereby obtaining a mixture comprising adsorbing agent-adsorbed prothrombin. In some embodiments, the medium is or comprises a liquid medium.

Thus, in some embodiments, the “medium” refers to a liquid (e.g., aqueous solution) in which the source of prothrombin and the BaSO₄ reagent are present upon contacting the BaSO₄ reagent and the source of prothrombin. In some embodiments, the solution is incubated at around room temperature for 1 to 6 hours after the contacting. By “around the room temperature” it is meant to refer to at least one temperature value within the range of 10 to 40° C., or 15 to 37° C., e.g., 10, 15, 20, 25, 30, 35, 37, or 40° C., including any value and range therebetween.

In some embodiments, the suitability of the adsorbing agent, e.g., BaSO₄ reagent, for use as a prothrombin adsorbent e.g., in a process for preparing thrombin, is indicated by the pro-coagulant activity of the adsorbing agent-adsorbed prothrombin being not greater than the pro-coagulant activity of normal mammalian plasma, e.g., normal human plasma. Specifically, in some embodiments, a sample of a given BaSO₄ reagent is contacted with a source of prothrombin to adsorb prothrombin therefrom to obtain BaSO₄-adsorbed prothrombin. Typically, the lower the pro-coagulant activity of the BaSO₄-adsorbed prothrombin, the more suitable the given BaSO₄ reagent is for use as a prothrombin adsorbent. In some embodiments, BaSO₄ reagents which yield eluates having a Non-Activated Partial Thromboplastin Time (NAPTT) ratio of 0.8 or less or clot are visually observed immediately (clotting occurring upon calcium addition and before clotting time can be recorded in a coagulator measurement machine) are considered less suitable for use in the preparation of thrombin.

As used herein, the term “pro-coagulant activity” refers to promotion of coagulation of blood. In some embodiments of the methods disclosed herein, evaluating pro-coagulant activity of the adsorbing agent-adsorbed prothrombin is of prothrombin while adsorbed to the adsorbing agent (e.g., BaSO₄ reagent).

Typically, but not exclusively, solvent detergent (SD) e.g., for viral inactivation treatment is used. SD refers to a process that inactivates enveloped or lipid-coated viruses by destroying their lipid envelope. The treatment may be carried out by the addition of detergents (such as Triton X-45, Triton X-100 or polysorbate 80) and solvents [such as tri(n-butyl) phosphate (TnBP), di- or trialkylphosphates]. The SD combination used to deactivate lipid coated viruses may be any solvent-detergent combination known in the art such as TnBP and Triton X-100; polysorbate 80 and Sodium cholate and other combinations.

The concentration of the solvent(s) detergent(s) used may be those commonly used in the art, for example as carried out as described in U.S. Pat. No. 5,094,960, or 4,789,545. The concentration of the solvent(s) detergent(s) used may be a combination of >0.1% TnBP and >0.1% Triton X-100. The concentration of the solvent(s) detergent(s) used may be a combination of e.g., 1% Triton X-100 and 0.3% TnBP. However, other solvent detergent (SD) combinations and suitable conditions will be apparent to any person versed in the art. In one embodiment, 0.5% to 1% Tween-80 and 0.15% to 0.3% TnBP is used for the SD treatment.

In some embodiments, the method further comprises a step of contacting the adsorbing agent e.g., BaSO₄ reagent, and the source of the protein of interest (e.g., prothrombin) under conditions allowing adsorption of the protein, e.g., prothrombin, from the source of prothrombin by the adsorbing agent e.g., BaSO₄ reagent, thereby adsorbing prothrombin into/onto the adsorbing agent e.g., BaSO₄ reagent.

In some embodiments, the conditions allowing adsorption of the protein, e.g., prothrombin from the source of prothrombin to the adsorbing agent e.g., BaSO₄ reagent, comprise a medium (e.g., solution) being at pH ranging from 7.4 to 8.6.

In one embodiment, the conditions allowing prothrombin adsorption to adsorbing agent (e.g., BaSO₄ reagent), in the preparation of thrombin comprise pH 7.4 to 8.6 and/or adsorbing agent (e.g., BaSO₄ reagent) being at a concentration range of about 0.5% to 22% (w/v) (e.g., about 1%), by total weight of the adsorbing agent and plasma. In some embodiments, the conditions comprise room temperature e.g., in the range of 20° C.-25° C.

In one embodiment, the adsorption of prothrombin by BaSO₄ is carried out in batch mode at room temperature e.g., at 25° C. for 2 hours at a pH 7.4-8.6. Some embodiments of the methods described herein enable a BaSO₄ reagent suitable for use as a prothrombin adsorbent.

In some embodiments of the methods disclosed herein, contacting the sample of the adsorbing agent (e.g., BaSO₄ reagent) and the source of prothrombin comprises adding from about 1% to about 22%, from about 0.5% to about 22%, or from about 0.5% to about 10% (w/v) of adsorbing agent (e.g., BaSO₄ reagent) to the source of prothrombin (e.g., harvested plasma). In some embodiments, about 0.5%, 1%, 2%, 3%, 4%, 5%, 6%, 7%, 8%, 9%, 10%, 11%, 12%, 13%, 14%, 15%, 16%, 17%, 18%, 19%, 20%, 21%, or 22% (w/v) BaSO₄ reagent is added, including any value and range therebetween.

Accordingly, in some embodiments, the medium comprises about 0.5 to 22% (w/w), or about 0.5 to 3% (w/w), adsorbing agent (e.g., BaSO₄ reagent), optionally about 1% adsorbing agent (e.g., BaSO₄ reagent), by weight.

The adsorption of prothrombin by the adsorbing agent (e.g., BaSO₄ reagent) may be carried out in batch mode or in a column packed with the adsorbing agent (e.g., BaSO₄ reagent).

As used herein, the term “BaSO₄ reagent” refers to a BaSO₄ reagent from a specified supplier. Different given BaSO₄ reagents may therefore be reagents provided by different suppliers, or different lots of reagent provided by the same supplier. In some embodiments, the BaSO₄ reagent is in the form of a powder. As used herein the term “powder” to a collection of particles. The particles may be of any configuration, shape or size as long as they are suitable for at least partially adsorbing the protein of interest, e.g., prothrombin.

In some embodiments, the BaSO₄ reagent is a reagent comprising at least 75% (w/w) BaSO₄, for example at least 80%, at least 85%, at least 90%, at least 95%, at least 97.5%, and even about 100% (w/w) BaSO₄.

The term “contacting” is used hereinthroughout in its broadest sense and refers to any type of combining action which e.g., brings the protein (e.g., prothrombin) source into sufficiently close proximity with adsorbent, e.g., BaSO₄, such that a binding interaction may occur between adsorbent, e.g., BaSO₄, and the prothrombin in the source. Contacting includes, but is not limited to, mixing, admixing and/or adding e.g., the source into the adsorbent (e.g., BaSO₄) or adding the adsorbent (e.g., BaSO₄) into the source.

In one embodiment, the adsorbent—e.g., BaSO₄— adsorbed protein is washed using a washing buffer, such as aqueous buffer. The washing step may wash away or dilute the impurities or inhibitors present in the sample or fraction containing the adsorbent—e.g., BaSO₄-adsorbed protein. As used herein, the term “wash away” may refer to remove the impurities or inhibitors completely or partially from the sample or fraction containing the adsorbent—e.g., BaSO₄— adsorbed protein using a buffer. As used herein, the term, “dilute” may refer to reduce the concentration of the impurities or inhibitors present in the sample, or in a fraction, containing the adsorbent—e.g., BaSO₄— adsorbed protein upon using the buffer. Thus, the washing step may result in complete or partial removal. The term “impurities” refers to materials (e.g., component or compound) in the medium e.g., the protein source, that are different from the protein of interest, or may react with the protein or its derivative e.g., fibrinogen in the case that the protein of interest is prothrombin.

In some embodiments, the washing buffer comprises sodium chloride and/or sodium citrate. In some embodiments, the washing step may be repeated several times, 2 to 10 times, or 2 to 5 times, e.g., 2, 3, 4, 5, 6, 7, 8, 9, or 10 times.

In some embodiments, passing the mixture in a pressure filter further allows separating off at least part of the adsorbent—e.g., BaSO₄— adsorbed prothrombin from the source (medium).

The term “eluting”, or any grammatical inflection thereof, is used herein to mean the release of the adsorbed protein of interest from the adsorbent, e.g., insoluble salt reagent, such as BaSO₄ reagent. Oftentimes, the term “elution” as disclosed herein is interchangeable with the term “desorption”. In some embodiments, this term relates to the release of at least 80%, at least 85%, least 90%, or at least 95%, of the adsorbed protein of interest from the adsorbent into the eluate. The elution may be carried out under certain elution conditions. Typically, but not exclusively, elution conditions include using a non-isocratic condition e.g., a solution or a condition different from the solution or condition used e.g., to load the adsorbent with the protein of interest, and/or different from the solution used in a previous step.

The method according to the invention comprises, in some embodiments, at least one elution step, typically with a non-isocratic solution.

The term “inhibitors” as used herein refers to materials (e.g., component or compound) that might reduce the activity of the protein of interest or might have an adversely affect.

In some embodiments, the method comprises at least two steps selected from: (i) centrifuging the medium, thereby obtaining a sediment comprising the adsorbent, e.g., BaSO₄ reagent, and/or the protein; (ii) washing the protein being at least partially adsorbed into/onto the adsorbent, e.g., BaSO₄ reagent, by a washing buffer, thereby removing therefrom impurities; and (iii) eluting a fraction comprising the protein (“protein containing fraction”) from the adsorbent—e.g., BaSO₄ reagent-adsorbed protein, e.g., using an elution buffer.

In some embodiments, the method comprises less than three centrifugation steps, no more than two centrifugation steps, or no more than one centrifugation step. Typically, but not exclusively, the disclosed method is devoid of using a centrifugation step.

In some embodiments, the term “sediment” is used herein to refer to a “pellet” or solid that is separated from the supernatant after the denser matter has been separated or removed from a liquid composition, for example, via the centrifugation or by using a filter press.

In some embodiments, the protein is eluted from the adsorbent—e.g., BaSO₄-adsorbed protein using an elution buffer, thereby obtaining an eluted protein-containing fraction.

The term “fraction” refers to a separable constituent e.g., comprising the protein of interest.

In one embodiment, the elution buffer comprises a calcium chelating salt. In some embodiments, the elution solution comprises a chelating salt. In some embodiments, the concentration of chelating salt in the elution solution ranges from about 0.2% (w/v) to about 4.4% (w/v) or from about 3.0% (w/v) to about 4.4% (w/v). In some embodiments, the chelating salt comprises sodium citrate. Accordingly, in some embodiments, the concentration of the sodium citrate in the elution solution is from about 0.2% (w/v) to about 4.4% (w/v) or from about 3.0% (w/v) to about 4.4% (w/v), e.g., 0.2%, 0.4%, 0.6%, 0.8%, 1%, 1.2%, 1.4%, 1.6%, 1.8%, 2%, 2.2%, 2.4%, 2.6%, 2.8%, 3%, 3.2%, 3.4%, 3.6%, 3.8%, 4%, 4.2%, or 4.4% (w/v), including any value and range therebetween.

In some embodiments the elution buffer has pH of between 6.3 to 7.4, e.g., 6.3, 6.4, 6.5, 6.6, 6.7, 6.8, 6.9, 7, 7.1, 7.3, or 7.4, including any value and range therebetween.

In some embodiments, the pH of the elution solution is not less than 6.1, not less than 6.2, or even not less than 6.3. In some embodiments, the pH of the elution solution is not more than 6.5, not more than 6.6 and even not more than 6.7, or between about pH 6.3 and 6.7. In some embodiments, the pH of the elution solution ranges from 6.3 to 7.4.

In some embodiments, one or both steps of: (i) washing the protein being at least partially adsorbed into/onto the adsorbent, e.g., BaSO₄ reagent (“the washing step”); and (ii) eluting a fraction comprising the protein from the adsorbent-adsorbed protein (“the eluting step”) is carried out by, or simultaneously to, the step of passing the medium in the pressure filter.

The term “simultaneously” used hereinthroughout does not necessarily mean that the whole relevant steps are carried out at same time, and may also refer, for example, to a case of first starting to carry out the washing step and immediately thereafter passing the medium in the pressure filter, or, for example, to a case of first passing the medium in the pressure filter and, immediately thereafter, carrying out the eluting step. In some embodiments, by “immediately” it is meant to refer to within 0 to 20 sec, 0 to 10 sec, or 0 to 2, e.g., 0, 1, 2, 3, 4, 5, 6, 7, 8, 9, 10, 11, 12, 13, 14, 15, 16, 17, 18, 19, or 20 sec, including any value and range therebetween.

In some embodiments, the method further comprises a step of diafiltrating the eluted protein-containing fraction using a diafiltration buffer.

By “diafiltrating”, or any grammatical derivative thereof, it is meant to refer to a dilution process that typically involves removal or separation of components (such as permeable molecules like salts, proteins, solvents etc.) of a solution based on their molecular size by using micro-molecule permeable filters, in order to obtain pure solution.

Non-limiting exemplary diafiltration buffer comprises glycine and/or sodium citrate. In some embodiments, the concentration of glycine in the diafiltration buffer ranges from about 0.5% (w/v) to about 1.5% (w/v). In some embodiments, the concentration of the sodium citrate in the diafiltration solution is about 1% (w/v).

In some embodiments, the diafiltration buffer has pH of between 6.5 and 7.5, e.g., 6.5, 6.6, 6.7, 6.8, 6.9, 7, 7.1, 7.3, 7.4, or 7.5, including any value and range therebetween.

In some embodiments, the diafiltrating step may be repeated several times, 2 to 10 times, or 2 to 6 times, e.g., 2, 3, 4, 5, 6, 7, 8, 9, or 10 times.

In some embodiments, the protein is prothrombin, and the method further comprises a step of providing conditions which allow conversion of the prothrombin, into thrombin, thereby obtaining a thrombin.

Accordingly, in an aspect of the present disclosure, there is provided a method of obtaining a thrombin from a source of prothrombin, the method comprising: (i) providing the medium comprising the prothrombin and adsorbent, e.g., BaSO₄ reagent, and performing pressure filtering, for example, by passing the liquid medium comprising the adsorbent, e.g., BaSO₄ reagent, and the source of the prothrombin in a pressure filter, e.g., so as to wash the adsorbent-adsorbed protein and/or to elute the protein from the adsorbent, thereby at least partially purifying the prothrombin, and (ii) providing conditions which allow conversion of the prothrombin into thrombin, thereby obtaining a thrombin.

Thus, in some embodiments, the prothrombin is at least partially adsorbed into/onto the adsorbent, e.g., BaSO₄ reagent. In some embodiments, the prothrombin contacting with the adsorbent, e.g., BaSO₄ reagent, triggers conversion into thrombin (i.e. conversion of prothrombin into its intermediates and/or into thrombin). This is a premature conversion that may compromise thrombin yields at the end of the production process. In some embodiments of the methods described herein, pro-coagulant activity occurs following the conversion of prothrombin into its intermediates and/or into thrombin. Such intermediates may be formed during the proteolytic conversion of prothrombin to thrombin. Non-limiting examples of intermediates are prothrombin and meizothrombin.

In one embodiment, the conditions which allow conversion of prothrombin into thrombin comprise subjecting the prothrombin to an activator such as calcium ions.

In exemplary embodiments, the activator comprises an activation buffer comprising calcium ions and glycine. In some embodiments a source for calcium ions is a calcium salt, such as calcium chloride. Calcium ions may be present at a concentration of 0.5% (w/v) to about 3% (w/v), or 0.75% to about 1.5%, e.g., 0.5%, 0.75%, 1%, 1.25%, 1.5%, 1.75%, 2%, 2.25%, 2.5%, 2.75%, or 3% (w/v), including any value and range therebetween. In some embodiments, the activation buffer has pH of between 6.5 to 7.5, e.g., 6.5, 6.6, 6.7, 6.8, 6.9, 7, 7.1, 7.3, 7.4, or 7.5, including any value and range therebetween.

In some embodiments, the obtained thrombin is present in a fraction, and the method comprises a step of passing the thrombin containing fraction in a filter to remove therefrom micro floc which may be present in the fraction. In some embodiments, the filter is a microfilter. As used herein, the term “microfilter” means a filter membrane with pore size of about 0.1 to about 10 microns, e.g., about 0.2 μm. Optionally, the filtered fraction is further dialyzed.

In some embodiments, the disclosed method is characterized by obtaining a thrombin yield of 70 to 130 IU per 1 ml of source of prothrombin, e.g., plasma, In some embodiments, the disclosed method is characterized by obtaining a thrombin yield of 70 to 130 IU per 1 ml of source of prothrombin, e.g., plasma, within 1 to 4 hours, or 2 to 4 hours from the onset of the process.

In some embodiments, the thrombin obtained by the disclosed method is characterized by activity of 3000 to 7000 IU/ml. In some embodiments, the thrombin obtained by the disclosed method is characterized by activity of 3000 to 6000 IU/ml. In some embodiments, the thrombin obtained by the disclosed method is characterized by activity of 4000 to 6000 IU/ml. In some embodiments, the thrombin obtained by the disclosed method is characterized by activity of 3000, 3500, 4000, 4500, 5000, 5500, 6000, 6500, or 7000 IU/ml, including any value and range therebetween.

Accordingly, in some embodiments, the thrombin obtained by the disclosed method is characterized by specific activity of 500 to 1500, 500 to 1200, 700 to 1500, or 700 to 1200 IU per mg protein.

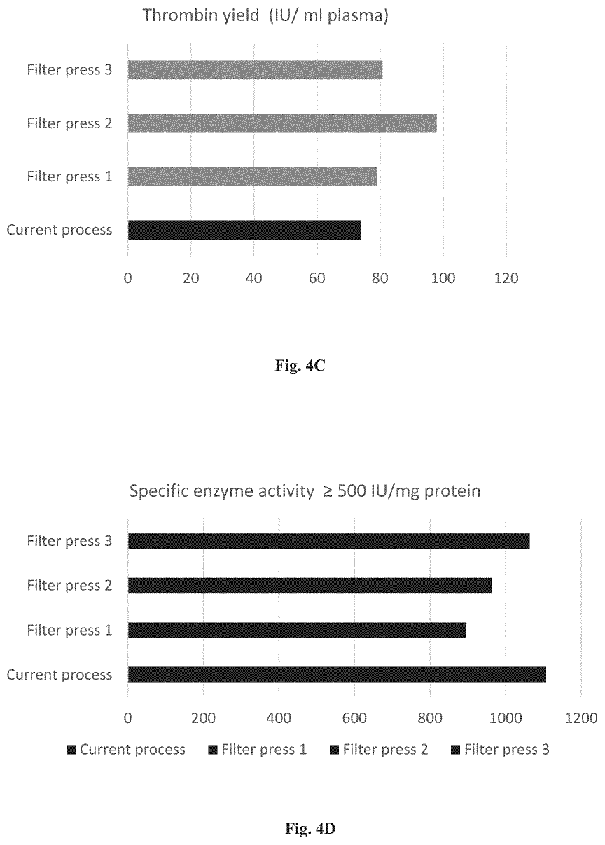

Reference is made to FIGS. 4A-D presenting a cooperative evaluation of protein content, thrombin activity, specific activity, and product yield of product which was made by filter press process and non-filter press process. At least no significant difference between the two processes has been evaluated according to T-test.

Various types of filter plates may be utilized in filter presses according to the present invention, for example and without limitation, plate and frame filter presses, recessed plate and frame filter presses, membrane filter presses, and (fully) automatic filter presses.

For example, one type includes a filtration chamber having a plate or a group of plates, with a plate which may be a chamber plate which includes recessed surfaces on opposite sides of the plate each of which serves to form a filter chamber with an adjacent plate when the plates are clamped together. A filter may cover each of these recessed surfaces, and may either be mounted on the plate by a gasket or is draped between two adjacent plates.

In some embodiments, a group of plates includes plates having a frame with a pair of oppositely disposed faces which are recessed inwardly. A permeable, non-permeable or semi permeable-membrane may be fixed to the frame and may extend across one of the recessed faces to define a pressure chamber therebetween.

In some embodiments, the membrane surface ranges from is 0.04 m² to 10 m², e.g., 0.04, 0.08, 0.12, 0.2, 0.4, 0.6, 0.8, 1, 1.2, 1.4, 1.6, 1.8, 2, 2.2, 2.4, 2.6, 2.8, 3, 3.2, 3.4, 3.6, 3.8, 4, 4.2, 4.4, 4.6, 4.8, 5, 5.2, 5.4, 5.6, 5.8, 6, 6.2, 6.4, 6.6, 6.8, 7, 7.2, 7.4, 7.6, 7.8, 8, 7.2, 7.4, 7.6, 7.8, 8, 8.2, 8.4, 8.6, 8.8, 9, 9.2, 9.4, 9.6, 9.8, or 10 m², including any value and range therebetween. In some embodiments, a couple or a few couples of membranes may be used.

In some embodiments, the membrane has microsized pores. The term “micro-sized” as used herein, unless specified, relates to an average particle size of between about 0.5 μm to about 100 μm, or, typically 0.5 to 5 μm, e.g., about 1 μm.

Thus, the medium may be pumped into the filter chambers formed between the filters of two adjacent plates, and the liquid medium may pass through the filter and then may be discharged through filtrate ports in the plates. The adsorbent-adsorbed protein according to the present disclosure may be trapped in the filter chamber and form a cake.

In some embodiments, the step of performing pressure filtering comprises passing the medium through a filtration chamber under pressure, the filtration chamber comprising a filter membrane, optionally selectively permeable or semi-permeable filter membrane. The term “semi-permeable membrane” means a membrane that is substantially selective based on a size or molecular weight. Thus, a semi-permeable membrane substantially passes a first molecular weight or size, while substantially blocking passage of second molecular weight or size, greater than the first molecular weight or size.

As described below, in exemplary embodiments, the filtration end may be kept partially closed at the initial stage of filtration, so that the back pressure of the filtration end is maintained at about 10 psi, as a certain back pressure may be helpful for the uniform distribution of barium sulfate precipitation on the membrane surface. As the filtration progresses, the feed port (inlet) pressure may increase gradually, so the back pressure needs to adjust also to ensure the pressure of feed port is less than 2 bar. Finally, the filtration process may be stopped when the inlet pressure is larger than 3.5 bar, e.g., 2 to 5 bar.

Accordingly, in some embodiments, the step of performing pressure filtering comprises passing the medium through a filtration chamber under a pressure, the filtration chamber comprising filter membrane. In some embodiment, the pressure ranges from 1.5 to about 4 bar, e.g., 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, or 4 psi, including any value and range therebetween. In some embodiments, the step of performing pressure filtering comprises passing the medium in the pressure filter and exerting a back pressure onto the membrane, the back pressure ranging from e.g., 5 psi to 15 psi, e.g., 5, 6, 7, 8, 9, 10, 11, 12, 13, 14, or 15 psi, including any value and range therebetween, thereby obtaining a uniform cake of the adsorbent (e.g., BaSO₄)-adsorbed protein in/on the filter membrane.

As described herein, passing the medium through a filtration chamber may be carried out by using a pump, i.e. pumping the medium into the system. In some embodiments, the medium is first circulated for 10 to 30 min e.g., 15 min in the system at low speed (e.g., 30 to 100 ml/min, such as about 70 ml/min) while keeping the pressure at the indicated pressure, e.g., at or below 1 bar (15 psi).

In some embodiments, the amount of protein source (e.g., plasma) which may be used according to the disclosed process ranges from 100 to 1500 kg, or in some embodiments 200 to 1000 kg, for example 100, 200, 300, 400, 500, 600, 700, 800, 900, 1000, 1100, 1200, 1300, 1400, or 1500 kg or more, including any value and range therebetween, depending for example on the number of the membranes used.

In some embodiments, the filter press process is characterized by a filtration capacity of at least 30 kg of the source of protein e.g., prothrombin, per m² membrane. In some embodiments, the filter press process is characterized by a filtration capacity of 30 to 200 kg of the source of protein e.g., prothrombin per m² membrane. In some embodiments, the filter press process is characterized by a filtration capacity of 30, 40, 50, 60, 70, 80, 90, 100, 110, 120, 130, 140, 150, 160, 170, 180, 190, or 200, including any value and range therebetween protein e.g., prothrombin, per m² membrane.

As used herein the term “about” refers to ±10%. Unless otherwise indicated, all numbers such as those expressing, for example, ratios, weight, amounts, pressure, temperatures, etc., are to be understood as being modified in all instances by the term “about”.

As used herein, and unless stated otherwise, the terms “by weight”, “w/w”, “weight percent”, or “wt. %”, which are used herein interchangeably describe the concentration of a particular substance out of the total weight of the corresponding mixture, solution, formulation or composition.

The terms “comprises”, “comprising”, “includes”, “including”, contains”, “containing”, “has”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “method” or “process”, which may be used hereinthroughout interchangeably, refer to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Herein, the disclosed process is referred to as: “the filter press process”; the currently known process used (prior to the development of the process of the invention) is interchangeably referred to as: “the current process”, “the manual process”, or “the non-filter press process”.

Both processes may be preceded by: Binding (mixing barium sulfate with plasma (1% m/m) to adsorb prothrombin on the barium sulfate); and thereafter in the manual process: Separation—by centrifugation to separate barium sulfate from plasma and collect barium sulfate cake; Washing—by washing buffer, crashing the sediment into small pieces manually, followed by centrifugation to separate solid from liquid (this step is repeated 3 times to remove impurity); and Elution—by adding elution buffer and crashing the sediment into small pieces manually, followed by centrifuging to separate solid from liquid. This step is repeated 5 times to collect prothrombin complex.

Materials and Equipment

Materials

The materials details are summarized in Table 1 below:

TABLE 1
Materials

| Material | Plasma | BaSO₄ |
|---|---|---|
| Supplier | Internal production | Qingdao Dongfeng |
| | | Chemical LTD. |
| Lot No. | 40151107 & 40160408 | 11140308 |
| Storage T | −20° C. | RT |

Equipment

The equipment details are summarized in Table 2 below:

TABLE 2
Equipment details

| Equipment | | |
|---|---|---|
| Name | Vendor | Type |
| Filter press | Ertelalsop | 4D-6-059 |
| Depth filter | Ertelalsop | M503P-89L |

### Example 1: Preliminary Tests

The purpose of the test is, inter alia, to investigate the feasibility of using the filter press to replace the manual operation of prothrombin purification (“the non-filter press process”)

A. First Set of Preliminary Tests:

In a first set of experiment, a preliminary experiment was conducted using a 1-micron pore size filter (Ertelalsop M503P-89L) to filtrate the suspension of BaSO₄ with plasma, observing the clarity of the filtration liquid, the flowrate of filtration, and the distribution of the barium sulfate cake on the depth filter membrane.

Feasibility Study—General Outline: after adding barium sulfate powder to the plasma, the prothrombin was sufficiently adsorbed, and then pumped the suspension into a filter press, and the barium sulfate particles were retained on the surface of the filter. The precipitated layer was washed with a washing buffer to remove excess impurities. After washing, the washing buffer was replaced with an elution buffer to elute the prothrombin from BaSO₄ and the eluted liquid from the filtrate comprising prothrombin was collected. The eluted liquid comprising prothrombin was concentrated, dialyzed to remove salt, and prothrombin was activated using CaCl₂. An outline of the filter press is presented in the flow chart presented in FIG. 1A. The process may be further continued to obtain thrombin as outlined in FIG. 1B and as detailed below.

Detailed Method

Binding: in exemplary procedures, 1823 g of plasma (batch number: 40151107, stored at −20° C.) were used after thawing for two hours in a 37° C. water bath (as noted below only 718 g of plasma passed through the membrane of filter press due to the membrane capacity of the membrane used). Next, 18.3 g of BaSO₄ solid (1:100 by weight ratio to the plasma) was added, and the mixture was stirred at room temperature for 2 hours.

Filtration: in exemplary procedures, the membrane in the filter press was washed by thoroughly circulating with 1 L of purified water (denoted as “PuW” or “PW”) at a pump speed of 250 rpm. The plasma barium sulfate mixture was filtered by a filter press, and the barium sulfate precipitate was hold by membranes (2 pieces of filter). The filtration end was closed a little bit at the initial stage of filtration, so that the back pressure of the filtration end was maintained at about 10 psi (a certain back pressure is helpful for the uniform distribution of barium sulfate precipitation on the membrane surface). As the filtration progressed, the inlet pressure increased gradually, so the back pressure needed to adjust also to ensure the pressure of feed port (inlet) was less than 2 bar. Finally, the filtration process was stopped when the inlet pressure was larger than 3.5 bar.

Through weighing the filtrated liquid, the filtration flux capacity of the membrane was calculated: there was 718 g of plasma that passed through the membrane of filter press, and with the surface of two pieces of filter being 200 cm² (the surface area of one piece of filter is 100 cm²), it gives a filtration capacity of the membrane of about 35.9 kg plasma/m².

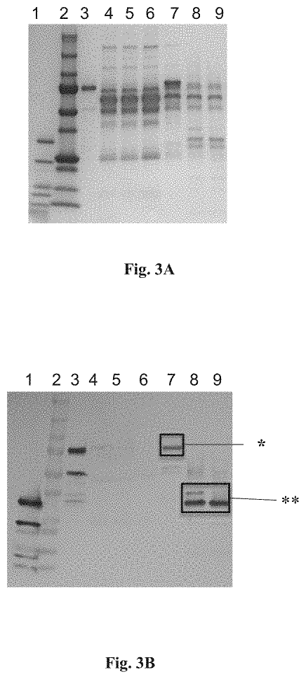

The filtration separation step showed that a depth filter with 1-micron pore size can effectively separate the barium sulfate from plasma mixture. The plasma passed through the filter membrane was clean, without visible white powder. However, when the filtration process progressed, barium sulfate precipitated continuously accumulated on the surface of the membrane, and the pressure of filtration increased. After the process completed, the filter press could be disassembled to check that barium sulfate was evenly distributed on the surface of the filter membrane (see FIG. 2).

Washing: in exemplary procedures, the BaSO₄ precipitates cakes were washed on the membrane with 600 ml washing buffer (0.45% NaCl+0.0025% Na-Citrate), followed by draining of the first 100 mL directly, and cycle for 15 minutes with the rest of 500 ml washing buffer. The washing step was repeated 5 times, and the fibrinogen (FIB) (which is impurity in this process) and the protein contents were tested for each sample from the washed liquid each time. The pump speed setting: 250 rpm. The results are as follows (Table 3):

TABLE 3
Washing Steps

| Washing | 1 | 2 | 3 | 4 | 5 |
|---|---|---|---|---|---|
| times | First | Second | Third | Fourth | Fifth |
| FIB | 0.31 | Not | not | not | not |
| (g/L) | | detected | monitored | monitored | monitored |
| Protein | 8.536 | 1.631 | 0.415 | 0.115 | 0.007 |
| content | | | | | |
| (mg/ml) | | | | | |

The volume ratio of the buffer to the plasma per wash in the non-filter press process was about 1:50 buffer to plasma. The filter press process needed a consideration as to the volume inside the plate frame, the volume of the pipe, so that the buffer could be circulated in the flow path. An amount of 600 ml washing buffer was used in each washing run. Sampled data showed that fibrinogen in the wash solution had not been detected after the second wash, and fibrinogen could be completely removed under this method. The protein content in the washing solution showed a downward trend. The protein content of the fifth washing solution was 0.007 mg/ml, and the protein impurity was close to zero.

Elution: in exemplary procedures, 500 ml elution buffers (3.0% Na-Citrate pH 6.5) were used, circulated for 15 minutes, followed by collecting the eluate liquid. The elution step was repeated for 5 times, providing a sample 1.0 ml liquid for protein content testing from the eluate liquid each time. The pump speed was set to 250 rpm. A total of approximately 2,500 ml of eluate liquid was collected and stored in a 4° C. refrigerator. The protein content results were as follows (Table 4):

TABLE 4
Elution steps

| | 1 | 2 | 3 | 4 | 5 |
|---|---|---|---|---|---|
| Elution times | First | Second | Third | Fourth | Fifth |
| (mg/ml) | 0.302 | 0.187 | 0.100 | 0.051 | 0.022 |
| Protein content | | | | | |

The ratio of the volume of the plasma to the elution buffer per elution in the manual non-filter press process was about 50:1 plasma volume/elution buffer volume. The use of the filter press process needed a consideration regarding the volume inside the plate frame, the volume of the pipe, so that the buffer that could be circulated in the flow path.

An amount of 500 ml elution buffer was used in each washing run.

The protein content in the washing solution showed a downward trend. The protein content of the fifth eluate was 0.022 mg/ml, and the amount of the protein eluted from barium sulfate was less and less.

Concentration, Diafiltration and Activation: in exemplary procedures, the eluate liquid which was collected in the previous step was concentrated to 41 ml. An amount of 200 mL diafiltration buffer (1.0% glycine pH 7.0) was then added for constant volume followed by dialysis 5 times (in total: 41×5 ml), followed by concentrating to 41 ml (comprising a solution of 1.0% glycine pH 7.0). The FIB was undetected in the concentrated eluate.

In exemplary procedures, 161 ml activated buffer (0.75% CaCl₂, 1.0% glycine pH 7.0) was thereafter added, activating for 8 hours at room temperature, followed by transferring to a 4° C. refrigerator for further activation (30 hours). The final solution had a volume of 202 ml.

The enzyme activity and protein content were measured after storage for 30 hours in the refrigerator, and the thrombin activity was 596 IU/ml. The protein content was 0.900 mg/ml. In exemplary procedures, the activated solution of the previous step was filtered by 0.2 μm filter, to remove the micro floc in the liquid, and then concentrated to it to around 40 ml, and then dialyzed with 120 ml purified water. Finally, 39.3 ml of thrombin bulk solution were obtained.

Thrombin activity and protein content were measured, and the enzyme activity was 1976 IU/ml, and the protein content was 2.430 mg/ml.

In the non-filter press process 20 L of eluent (comprising the prothrombin) were collected, followed by concentrating to 4 L (i.e. a concentration factor of 5); the amount of initial plasma per batch was approximately 220 L, and the volume ratio of plasma (220 L)/thrombin bulk (4 L) was about 55. For a comparative purpose, in the filter press process similar ratios of about 55 between the plasma and bulk were also selected.

Due to the limitation of the ultrafiltration (“UF”) device used in exemplary procedures (for the concentrating of prothrombin), the circulation volume is set to at least about 30 Therefore, the amount of plasma to be filtered by the filter press experiment was more than 1650 ml (i.e. more than 30 ml×55).

Comparison of filter press to the manual process: in the filter press preliminary experiment, the amount of plasma processed by the filtration step was only 718 ml, and the theoretical volume of the bulk solution should be 13 ml (i.e. 718/55). However, as noted above, in exemplary procedures, the actual experiment was limited by the ultrafiltration equipment dead volume, and the final bulk volume of the bulk solution was 39.3 ml. Thus, the thrombin activity of the final bulk solution was 1976 IU/ml, and the protein content was 2.430 mg/ml. However, this does not affect the yield and thrombin specific activity calculations.

The parameters of the filter press process are as follows:

Yield=1976 IU/ml×39.3 ml/718 ml plasma=108 IU/ml plasma;

Specific activity=1976 IU/ml/2.430 mg/ml=813 IU/mg protein.

The non-filter press process:

Yield—74 IU/ml plasma, specific activity—1107 IU/mg protein.

Hence, through the comparison of these two-group data, the filter press process can make the product which have at least approximate result to the non-filter press process product.

B. Second Set of Preliminary Tests:

In an additional exemplary set of preliminary experiments, different porcine plasma (2 kg) was treated with solvent detergent (1% Tween-80 and 0.3% TnBP) for 1 hour at 25° C. BaSO₄ was added to a final concentration of 1% w/w (20 gr) and mixed for 2 hours at room temperature (RT). The suspension was centrifuged at 6000×g for 15 min at 4° C., the supernatant discarded and the BaSO₄ was kept frozen at ≤−30° C. until use.

The filter press system 50 cm² membrane (50P of PALL) was assembled with 2 membranes, 2 papers, 2 plates, 1 collecting frame and 1 blanking head. The BaSO₄ sediment was thawed and resuspended in 350 ml of washing buffer, in excess relative to a non-filter press process, to ensure efficient washing of all unbound components. The system was first washed by circulating the buffer at low speed to remove air from the system. The solution was then pumped into the system and circulated for 15 min in the system at low speed (70 ml/min) while keeping the pressure at or below 1 bar (15 psi). Protein level (absorbance 280-320 nm) was measured periodically during circulation of the wash buffer in the outlet pipe and the buffer tank until equilibrium was reached. The wash solution was pumped out and drained from the system.

Next, elution buffer (200 ml) was pumped into the system and circulated for 15 min Protein level (absorbance 280-320 nm) was measured periodically during circulation of the elution buffer in the outlet pipe and the buffer tank until equilibrium was reached. The elution buffer was pumped out of the system and collected in a clean vessel.

In order to verify that the eluted prothrombin can be converted to thrombin, 12 ml of the eluted sample were concentrated to 2.4 ml by centrifugation in Centricon® centrifugal filter unit at 4,800×g for 13 min at 25° C. PuW was added up to 12 ml and another centrifugation step was performed. PuW addition and centrifugation were repeated one more time. Next, an activation buffer was added (CaCl₂-Glycine) up to 12 ml and the sample was incubated for 8 hours at 25° C. followed by 60 hours at 4-8° C. for activation.

Prothrombin (FII) activity in the samples was evaluated using Diagnostica Stago Inc. reagents and clotting machine and calculated relative to normal human plasma.

Western blot assay for detection of prothrombin and thrombin in test samples was performed using sheep anti human thrombin (Affinity biologicals) as primary antibody and donkey anti-sheep IgG Alk. Ph. (Sigma) as secondary antibody.

Results: Prothrombin (FII) activity (Table 5) was measured in different fractions from two independent filter press runs compared with lab scale samples of the non-filter press process, as well as in-process samples obtained from a batch of the manufacturing process. The results indicate that elution fractions from both filter press runs had similar level of prothrombin recovered from the starting plasma (21-23%). These values were similar to the level found in lab scale samples produced according to the non-filter press capacity process (23%), and within the same order of magnitude as the in-process samples obtained from a full-scale production batch in the non-filter press process (32%) (see Table 5 below).

TABLE 5
FII activity of in-process samples (% relative to normal human plasma)

| | | | | | | | Nonfilter | |
|---|---|---|---|---|---|---|---|---|
| | | | | | Non-filter | | press in- | |
| | Filter | | Filter | | press process | | process | |
| | press #1 | | press #2 | | lab scale | | samples | |
| | Weight | FII | Weight | FII | Weight | FII | Weight | FII |
| Fraction | (Kg) | (% IU/ml) | (Kg) | (% IU/ml) | (Kg) | (% IU/ml) | (Kg) | (% IU/ml) |
| Plasma | 2 | 45 | 2 | 66 | 0.2 | 73 | 220 | 47 |
| Elution | 0.2 | 105 | 0.2 | 141 | 0.0075 | 450 | 20 | 165 |

| | | | | |
|---|---|---|---|---|
| Recovery | 23% | 21% | 23% | 32% |

Table 6 below shows the values of parameters used in this filter press feasibility study relative to non-filter press process. The table also shows theoretical calculated parameters based on the lab scale system adjusted to non-filter press production scale as well as five-fold scale up from old scale.

TABLE 6
Parameters of filter press feasibility study

| | Non-filter | | Lab scale | | Filter Press | | Filter press | |
|---|---|---|---|---|---|---|---|---|
| | press | | Filter press | | (36″) old | | scale up x5 | |
| Parameter | process | | (4″) | | scale* | | (36″)* | |
| Plasma | 200 | Kg | 2 | Kg | 200 | Kg | 1000 | Kg |
| BaSO₄ | 2 | Kg | 20 | gr | 2 | Kg | 10 | Kg |
| Filter area | N/A | | 0.0156 | m² | 1.56 | m² | 7.8 | m² |
| Cassettes (2 | N/A | | 1 | | 2 × 1.34 m² | | 6 × 1.34 m² | |
| membranes) | | | | | | | | |
| BaSO₄ per | N/A | | 1282 | gr/m² | 746 | gr/m² | 1244 | gr/m² |
| filter area | | | | | | | | |
| Wash buffer | 12 | Kg | 0.35 | Kg | ~30-50 Kg | | ~150-250 Kg | |
| (total) | | | | | | | | |
| Elution buffer | 20 | Kg | 0.2 | Kg | ~20 | Kg | ~100 | Kg |
| (total) | | | | | | | | |

| Process | 5 (wash) + | 3 | −3 | −3 |
|---|---|---|---|---|
| duration | 13 (elution) | | | |
| (hours) | | | | |

*Theoretical calculation

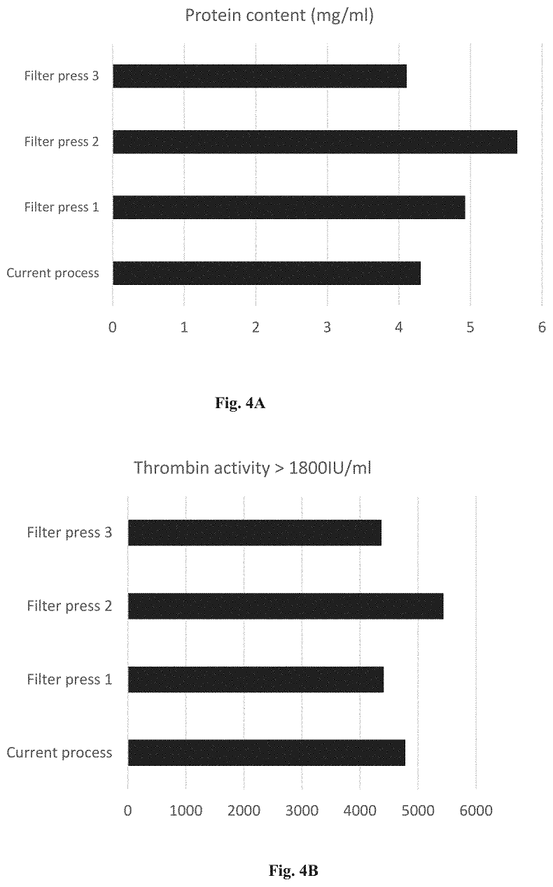

FIGS. 3A-B show SDS PAGE Coomassie staining (FIG. 3A) and Western blot (FIG. 3B) for prothrombin/thrombin in different fractions from the lab scale study compared with prothrombin and thrombin standards. The results show the presence of a band corresponding with prothrombin in the elution fraction (lane 7) and alpha-thrombin in the activated samples (lanes 8, 9). These results confirm the presence of active prothrombin in elution fraction obtained using the filter press system. Taken together, it can be concluded that comparing the filter press process to the non-filter press manual washing and/or elution process, the final product quality and yield is similar. But the filter press process has a lower risk of contamination, it can be automated, does not require centrifugation, and is easy to scale up, thereby it is feasible to use filter press process to replace manual process of thrombin production.

### Example 2: High-Scaled Feasibility Experiments

According to the results of the first set of preliminary experiments, the amount of plasma filtered by the two membranes was small, and the filter was increased to 4 pieces in the feasibility experiment. At the same time, in order to improve the washing effect, the first 300 ml of the washing buffer was directly drained in each circulation run, and the rest of 300 ml was circulated for 15 minutes. The feasibility experiment was repeated 3 times, and in the 3^{rd} experiment, the number of washing times was adjusted to 4 times. The rest of the operation was consistent with the pre-experiment. The experimental parameters and the results are shown in Table 7 below, and are further illustrated in FIGS. 4A-D:

TABLE 7
Experimental parameters and results-high scaled process

| | Non-filter | | | |
|---|---|---|---|---|
| | press | | | |
| Parameter | Process | Filter Press Process | | |
| Run order | N/A | 1 | 2 | 3 |
| Plasma (kg) | 220 | 1.728 | 1.684 | 1.784 |
| plasma lot no. | N/A | 40160608 | 40160608 | 40151107 |
| | | | | 40160408 |
| BaSO₄ (g) | 2200 | 17.3 | 16.8 | 17.8 |
| Filter (Ertelalsop) | N/A | M503P-89L | M503P-89L | M503P-89L |
| Filter amount | N/A | 4 | 4 | 4 |
| Washing Buffer in each cycle | 4000 | 600 | 600 | 600 |
| (ml) | | | | |
| Washing times | 3 | 5 | 5 | 4 |
| Elution Buffer each cycle (ml) | 4000 | 500 | 500 | 500 |
| Elution times | 5 | 5 | 5 | 4 |
| Total volume of collection (ml) | 20000 | 2500 | 2500 | 2000 |
| UF1 Concentration to (ml) | 4000 | 39.4 | 45 | 40.5 |
| Activation buffer (ml) | 16000 | 157.6 | 180 | 162 |
| Activated solution volume (ml) | 20000 | 196 | 221 | 202.5 |
| Protein content (mg/ml) | N/A | 1.499 | 1.246 | 0.781 |
| Thrombin activity (IU/ml) | N/A | 1135 | 857.5 | 751 |
| UF2 Concentration to (ml) | 4000 | 31 | 30.3 | 33 |
| Protein content (mg/ml) | 4.3 | 4.921 | 5.65 | 4.104 |
| Thrombin activity > 1800 IU/ml | 4781 | 4410 | 5439 | 4370 |
| Thrombin yield (IU/ml | 74 | 79 | 98 | 80.8 |
| plasma) | | | | |
| Specific enzyme activity ≥ | 1107 | 896 | 963 | 1064 |
| 500 IU/mg protein | | | | |

The protein content and thrombin activity data, presented in Table 7 and visualized in FIGS. 4A-D, show that the quality of thrombin obtained from the filter press process three times is similar to the results of the non-filter press process. The average value of the 3 runs protein content was 4.892 mg/ml, and the average thrombin activity was 4740 IU/ml. The protein content of the non-filter process in the production was 4.300 mg/ml, the thrombin activity is 4781 IU/ml. The acceptance standard of thrombin activity of production is ≥1800 IU/ml.

Thrombin specific activity: the data show that the washing step of the filter press can remove the impure protein. The average value of the three-run enzyme specific activities was 974 IU/mg protein, the production data was 1107 IU/mg protein, and the acceptance standard was greater than 500 IU/mg protein.

Thrombin yield: the thrombin yield is calculated based on the used plasma. The data show that the filter press elution step can elute the prothrombin from BaSO₄. The average of 3 experiments was 86 IU Thrombin/ml plasma, and the production statistics data of the non-filter process were 74 IU Thrombin/ml plasma.

Membrane area: the feasibility experiment scale is approximately 1/125 of the non-filter press production scale. The membrane used in this procedure was approximately 0.04 m². Based on this calculation, the area of the membrane used for the production scale is estimated to be 5 m². However, the loading capacity of the membrane can be further optimized, and the membrane area used may be reduced.

Washing buffer volume: the total amount of washing buffer used in the third filter press experiment was 2400 ml, and with scaling it up to a production scale, the washing buffer volume is estimated to be 300 L. However, in production scale, the total volume of the washing buffer may be reduced by optimizing the dead volume of the equipment, the flow path, and by optimizing the washing process parameters.

Elution buffer volume: the total amount of washing buffer used in the third filter press experiment was 2000 ml, and scaling it up to a production scale, the washing buffer volume is estimated to be 250 L. However, in production scale, the total volume of the washing buffer may be reduced by optimizing the dead volume of the equipment, the flow path and optimizing the washing process parameters.

Process time: the manual wash/elution process time is approximately 16 hours, while the filter press wash/elution process time is approximately 2 to 4 hours.

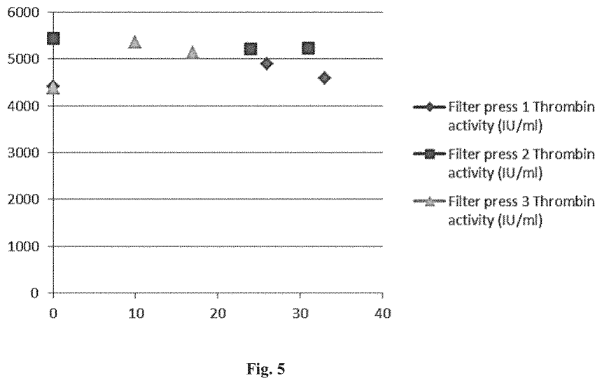

Prothrombin frozen storage: in exemplary procedures, prothrombin bulk liquid obtained from each of the above three feasibility experiments, 1 ml/per, were frozen in a refrigerator at −20° C., and the thrombin activity was measured after thawing at room temperature. The results of frozen time and thrombin activity are shown in FIG. 5 presenting scatter diagram of thrombin activity frozen under −20° C. It is shown that the thrombin activity remains stable after the thrombin bulk solution was frozen in a refrigerator under −20° C. for 30 days.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of purifying a protein of interest from a medium comprising an insoluble adsorbent, the method comprising providing said medium comprising the protein, the protein being at least partially adsorbed into/onto the adsorbent, and performing pressure filtering to wash the adsorbent-adsorbed protein and/or to elute the protein from the adsorbent, thereby at least partially purifying the protein.

2. The method of claim 1, wherein the adsorbent comprises an insoluble salt.

3. The method of claim 2, wherein the insoluble salt comprises aluminium hydroxide.

4. The method of claim 2, wherein the insoluble salt comprises an insoluble alkaline earth metal salt.

5. The method of claim 4, wherein the insoluble alkaline earth metal salt is or comprises a BaSO₄ reagent.

6. The method of any one of claims 1 to 5, wherein the medium comprises a source of said protein.

7. The method any one of claims 1 to 6, wherein the medium is a liquid medium.

8. The method of any one of claims 1 to 7, wherein the protein comprises prothrombin.

9. The method of any one of claims 1 to 8, wherein the medium comprises a source of prothrombin.

10. The method of any one of claims 1 to 9, comprising performing pressure filtering to wash the adsorbent-adsorbed protein and to elute the protein from the adsorbent, optionally the adsorbent comprising BaSO₄.

11. The method of any one of claims 1 to 10, wherein the adsorbent-adsorbed protein is washed using a washing buffer.

12. The method of any one of claims 1 to 11, comprising one or more steps selected from: (i) centrifuging the medium, thereby obtaining a sediment comprising said adsorbent and/or said protein; (ii) washing the protein being at least partially adsorbed into/onto the adsorbent, optionally being a BaSO₄ reagent, by a washing buffer, thereby removing therefrom impurities; and (iii) eluting a fraction comprising said protein from the adsorbent-adsorbed protein, using an elution buffer.

13. The method of claim 12, comprising step (ii) and (iii), wherein at least one step from steps (ii) and (iii) is carried out by, or simultaneously to the step of performing pressure filtering, optionally being carried out by passing the medium in a pressure filter.

14. The method of any one of claims 1 to 13, wherein the adsorbent is in the form of powder.

15. The method of any one of claims 1 to 14, wherein the pressure filtering is carried out by a filter press.

16. The method of any one of claims 1 to 15, wherein the protein is prothrombin, and wherein the source of prothrombin is selected from the group consisting of blood plasma or a plasma fraction.

17. The method of claim 16, wherein the plasma comprises oxalated plasma.

18. The method of claim 16 or 17, wherein the source of prothrombin comprises plasma harvested from a mammal.

19. The method of claim 18, wherein the mammal is selected from the group consisting of a human, an equine, a bovine and a porcine.

20. The method of any one of claims 16 to 19, wherein the source of prothrombin comprises porcine plasma.

21. The method of any one of claims 9 to 20, further comprising a step of contacting the adsorbent (e.g., BaSO₄ reagent) and the source of prothrombin under conditions allowing adsorption of prothrombin from the source of prothrombin into/onto the adsorbent (e.g., BaSO₄ reagent), thereby adsorbing prothrombin into/onto the adsorbent (e.g., BaSO₄ reagent).

22. The method of claim 21, wherein the conditions allowing adsorption of prothrombin from the source of prothrombin into/onto the adsorbent (e.g., BaSO₄ reagent) comprise the medium having pH ranging from 7.4 to 8.6.

23. The method of any one of claims 1 to 22, wherein the step of performing pressure filtering comprises passing the medium through a filtration chamber under pressure, the filtration chamber comprising filter membrane.

24. The method of claim 23, wherein the pressure ranges from 1.5 to about 4 bar.

25. The method of claims 1 to 24, wherein the step of performing pressure filtering comprises passing the medium in a pressure filter and exerting a back pressure onto said membrane, said back pressure ranging from 5 psi to 15 psi, thereby obtaining a uniform cake of the adsorbent-adsorbed protein in/on said filter membrane.

26. The method of any one of claims 23 to 25, wherein the filter membrane is characterized by a filtration capacity of at least 30 kg of the source of prothrombin per m².

27. The method of any one of claims 23 to 26, wherein the filter membrane has micro sized pores.

28. The method of any one of claims 1 to 27, wherein the medium comprises about 0.5 to 3% (w/w) BaSO₄ reagent, optionally about 1%.

29. The method of any one of claims 11 to 28, wherein the washing step is repeated 2 to 6 times.

30. The method of any one of claims 11 to 26, wherein the washing buffer comprises sodium chloride and/or sodium citrate.

31. The method of any one of claims 1 to 30, wherein the protein is eluted from the adsorbent-adsorbed protein using an elution buffer, thereby obtaining an eluted protein-containing fraction.

32. The method of claim 31, wherein the elution buffer comprises a calcium chelating salt, optionally at pH of about 6.3 and 7.4.

33. The method of claim 32, wherein the calcium chelating salt comprises sodium citrate.

34. The method of claim 33, wherein the concentration of sodium citrate ranges from about 3% (w/v) to about 4.4% (w/v).

35. The method of any one of claims 1 to 34, further comprising a step of concentrating the eluted protein—optionally prothrombin—containing fraction.

36. The method of any one of claims 1 to 35, wherein the concentrating is carried out by diafiltrating the eluted protein-containing fraction in a diafiltration buffer.

37. The method of claim 36, wherein the diafiltration buffer comprises glycine.

38. The method of any one of claim 36 or 37, wherein the diafiltrating step is repeated 2 to 6 times.

39. The method of any one of claims 1 to 38, wherein a total time duration of the step of washing the adsorbent-adsorbed protein and/or the step of eluting the protein from the adsorbent is less than 16 hours, optionally 2 to 6 hours.

40. The method of any one of claims 1 to 39, wherein the protein is prothrombin, and wherein the method further comprises a step of providing conditions which allow conversion of the prothrombin into thrombin, thereby obtaining a thrombin.

41. A method of obtaining a thrombin from a source of prothrombin, the method comprising: (i) passing a liquid medium comprising: adsorbent, optionally a BaSO₄ reagent, and a source of said prothrombin, in a pressure filter, thereby at least partially separating and/or purifying the prothrombin from said medium, and (ii) providing conditions which allow conversion of the prothrombin into thrombin, thereby obtaining the thrombin.

42. The method of claim 41, wherein the adsorbent, optionally a BaSO₄ reagent, at least partially adsorbs said prothrombin.

43. The method of any one of claims 40 to 42, wherein the conditions which allow conversion of prothrombin into thrombin comprise subjecting the prothrombin to an activator, optionally comprising calcium ions.

44. The method of any one of claims 40 to 43, wherein the thrombin is obtained in a fraction, and the method comprises a step of passing the thrombin containing fraction in a filter to remove therefrom micro floc.

45. The method of any one of claims 40 to 44, characterized by obtaining a thrombin in a yield of 70 to 130 IU per 1 ml of source of prothrombin, optionally plasma.

46. A thrombin obtained by the method of any one of claims 40 to 45.

47. The thrombin of claim 46, characterized by activity of 4000 to 6000 IU/ml.

48. The thrombin of claim 46 or 47, characterized by specific activity of 700 to 1200 IU per mg protein.
